(19) United States
(12) Patent Application Publication
(10) Pub. No.: US 2024/0416105 A1
(43) Pub. Date: Dec. 19, 2024

(54) VENTRICULAR ASSIST SYSTEM

(71) Applicant: NATIONAL TSING HUA UNIVERSITY, Hsinchu City (TW)

(72) Inventor: Ting-Wei WANG, Hsinchu City (TW)

(73) Assignee: NATIONAL TSING HUA UNIVERSITY, Hsinchu City (TW)

(21) Appl. No.: 18/588,253

(22) Filed: Feb. 27, 2024

(30) Foreign Application Priority Data
Jun. 14, 2023 (TW) 112122273

(51) Int. Cl. A61M 60/183

(52) U.S. Cl. CPC A61M 60/183; A61M 2205/04; A61M 2205/103; A61M 2205/3303; A61M 2205/3317; A61M 2205/3365; A61M 2205/3561; A61M 2205/50; A61M 2210/125

(57) ABSTRACT

A ventricular assist system comprises a ventricular state sensor and a ventricular assist device coupled to the ventricular state sensor. The ventricular state sensor includes a coil and a control module coupled to the coil. Wherein the control module is configured to drive the coil to perfume an eddy current induction measurement to a ventricle of the subject, and derive at least one ventricular state information of the subject through the eddy current induction measurement. Wherein the ventricular assist device receives the at least one ventricular state information to adjust at least one ventricular control parameter of the ventricular assist device.

### TECHNICAL FIELD

The present invention relates to a ventricular assist system, in particular, a ventricular assist system with a ventricular state sensor and a ventricular assist device.

### BACKGROUND

Ventricular assist device is a mechanical cardiac assist pump used to treat patients with end-stage heart failure and prolong their lifespan to extend the life time for waiting heart transplantation. The main mechanism of the ventricular assist device is the usage of a driving motor installed in a failed ventricle to connect the aorta by a connecting tube. Therefore, the blood may be transported from the failed ventricle to the aorta and then to the vascular system of the whole body.

The ventricular assist device will provide an opportunity for heart failure patients to extend their lifespan before they have a transplantable heart. However, after installing the ventricular assist device, monitoring ventricular blood flow is a crucial issue for patient's safety. Therefore, the patient installed with the ventricular assist device needs to regularly return to hospital and track whether there is a significant improvement of the ventricular contraction/relaxation through instruments such as a cardiac ultrasound, and whether the pumping speed of the driving motor of the ventricular assist device should be adjusted is evaluated according to the tracking result. However, the cardiac ultrasound is only available in medical institutions and requires professional operators to operate. For the patient installed with the ventricular assist device, regularly returning to hospital for ultrasound diagnosis and treatment is quite time-consuming and costly. Besides, the current ventricular status of the patient is impossible to be known immediately. If the patient is uncomfortable or encounters any adverse reactions, the inability to immediately adjust the ventricular assist device based on the current ventricular state will affect the patient's recovery.

For patients installed with the ventricular assist device, a real-time detection for ventricular functions and a dynamic adjustment to the ventricular assist device will effectively improve the postoperative ventricular recovery and significantly reduce the risk of death. Therefore, a widely available and easy-to-use measurement and a feedback mechanism for a ventricular assist system will be a major development issue in the technical field.

### SUMMARY

One of the objects of the present invention is to provide a ventricular assist system for real-time detecting the ventricular contraction/relaxation and dynamically adjusting the parameter of the ventricular assist system.

The preset invention provides a ventricular assist system. The ventricular assist system includes a ventricular state sensor and a ventricular assist device coupled to the ventricular state sensor. The ventricular state sensor includes a coil and a control module coupled to the coil. The control module is configured to drive the coil to perform an eddy current induction measurement on a ventricle of the subject, and derive at least one ventricular state information of the subject through the eddy current induction measurement. The ventricular assist device receives the at least one ventricular state information to adjust at least one ventricular control parameter of the ventricular assist device.

As described above, the ventricular assist system of the present invention will detect the ventricular information (such as ventricular contraction or relaxation) of the subject through the ventricular state sensor by using an eddy current sensing mechanism. The ventricular information is feedback to the ventricular assist device, so that the ventricular assist device can adjust the at least one ventricular control parameter based on the current ventricular state of the subject. Compared to conventional technologies, the ventricular assist system of the present invention can be fabricated by electronic processes to reduce the size and the cost of the ventricular assist system. The mechanism of the eddy current induction measurement will provide a non-invasive or preferably a non-contact measurement. Accordingly, the ventricular assist system with smaller size and lower cost has the potential to achieve widespread use and home use.

### BRIEF DESCRIPTION OF THE DRAWINGS

The accompanying drawings are presented to help describe various aspects of the present invention. In order to simplify the accompanying drawings and highlight the contents to be presented in the accompanying drawings, conventional structures or elements in the accompanying drawings may be drawn in a simple schematic way or may be omitted. For example, a number of elements may be singular or plural. These accompanying drawings are provided merely to explain these aspects and not to limit them.

FIG. 1 is a schematic diagram of the ventricular assist system according to an embodiment of the present invention.

FIG. 2 is a schematic diagram of the measurement of the ventricular assist system according to an embodiment of the present invention.

FIG. 3 is an exemplary block diagram of the signal generating unit according to an embodiment of the present invention.

FIG. 4 is an exemplary schematic diagram of the data correction and establishment of the ventricular assist system according to an embodiment of the present invention.

FIG. 5 is a schematic diagram of the ventricular state sensor with a depth detection component according to an embodiment of the present invention.

FIG. 6 is a schematic diagram of the ventricular state sensor with the isolation unit according to an embodiment of the present invention.

FIGS. 7 to 8 are schematic diagrams of the ventricular state sensor configured to output leading electromagnetic signals according to an embodiment of the present invention.

### DETAILED DESCRIPTION

Any reference to elements using terms such as “first” and “second” herein generally does not limit the number or order of these elements. Conversely, these names are used herein as a convenient way to distinguish two or more elements or element instances. Therefore, it should be understood that the terms “first” and “second” in the request item do not necessarily correspond to the same names in the written description. Furthermore, it should be understood that references to the first element and the second element do not indicate that only two elements can be used or that the first element needs to precede the second element. Open terms such as “include”, “comprise”, “have”, “contain”, and the like used herein means including but not limit to.

The term “coupled” is used herein to refer to direct or indirect electrical coupling between two structures. For example, in an example of indirect electrical coupling, one structure may be coupled with another structure through a passive element such as a resistor, a capacitor, or an inductor.

In the present invention, the term such as “exemplary” or “for example” is used to represent “giving an example, instance, or description”. Any implementation or aspect described herein as “exemplary” or “for example” is not necessarily to be construed as preferred or advantageous over other aspects of the present invention. The terms “about” and “approximately” as used herein with respect to a specified value or characteristic are intended to represent within a value (for example, 10%) of the specified value or characteristic.

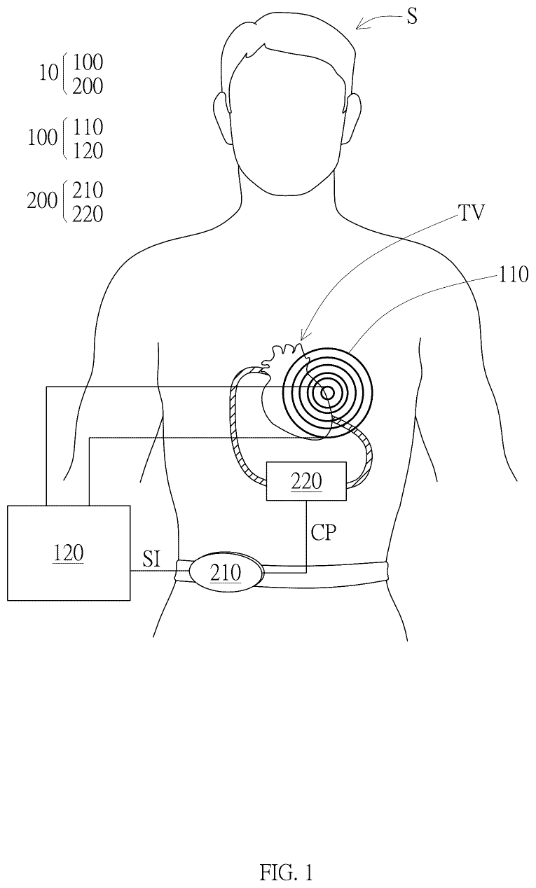

Referring FIG. 1, FIG. 1 illustrates the ventricular assist system 10 includes the ventricular state sensor 100 and the ventricular assist device 200 coupled to the ventricular state sensor 100. The ventricular state sensor 100 includes the coil 110 and the control module 120 coupled to the coil 110. The control module 120 is configured to drive the coil 110 to perform the eddy current induction measurement on the ventricle of the subject (TV), and derive at least one ventricular state information (SI) of the subject(S) through the eddy current induction measurement. The ventricular assist device 200 receives the at least one ventricular state information (SI) to adjust the at least one ventricular control parameter (CP) of the ventricular assist device 200.

The ventricular assist device (VAD) 200 is an electromechanical device configured to assist in cardiac circulation. The ventricular assist device 200 can be used to replace the function of the failed heart of the subject partially or completely. The ventricular assist device 200 can function to assist the heart for pumping blood. The ventricular assist device 200 can be designed to assist the right ventricle (RVAD), left ventricle (LVAD), or both ventricles (BiVAD). The ventricular assist device 200 includes the controller 210 and the blood flow assist pump 220 connected to the controller 210.

The coil 110 of the ventricular state sensor 100 can be a conductive wire formed on a substrate. More specifically, the conductive wire formed on the substrate can be formed by conventional manufacturing techniques such as etching, engraving, and photolithography. The conductive wire has at least one radiation portion configured to transmit electromagnetic signals, and receive feedback electromagnetic signals. The coil 110 can be, but not limited to, a single turn coil, a multi-turn coil, or a helical coil. In addition, the coil on the substrate can be a planar coil, for example, forming conducting wire on one layer of the substrate. On the other hand, the coil on the substrate can also be a three-dimensional coil, for example, forming a coil pattern with a conductive wire of at least two layers on the substrate. By using a conventional circuit manufacturing method to produce the coil 110, the yield and consistency of the coil 110 can be effectively improved. Furthermore, the coil 110 can be easily integrated with other circuit components and modules. Alternatively, the coil 110 can be a separate component without the need to be arranged on a substrate. For example, the coil 110 is a coil wound with enameled wire (for example only, not to limit the material of the coil 110). The types of the coil 110 can be selected from different radiation parts, materials, turns, shapes, etc. according to the purpose.

The control module 120 is coupled to the coil 110. For example, the control module 120 can be an independent module coupled to the coil 110. More specifically, the independent control module 120 can be a programmable or controllable module or device such as a computer, tablet, industrial computer, instrument, FPGA, microprocessor, etc. The control module 120 arranged as an independent control module will suit for different computing capabilities according to different requirements. For instance, when a high computing capability or a high level of regulatory/safety requirements need to be met, a component with advanced computing capability can be selected as the control modules 120. On the contrary, when lightweight and easy to carry are needed, a highly integrated component such as system on a chip (SOC) or application specific integrated circuit (ASIC) can be selected as the control modules 120.

The control module 120 can be coupled to the coil 110 on a substrate or by substrates. For example, the control module 120 and the coil 110 can be arranged on the same substrate or on different substrates. More specifically, the coil 110 can be formed on a substrate and connected to the control module 120 arranged on the same substrate by conductive wires formed on the substrate. The active/passive components required for the control module 120 can be arranged on the substrate, for example, by welding. In this way, the control module 120 and the coil 110 can be integrated into a uni-part that is easy to carry/wear, such as a card form. This can improve the overall integrity of the ventricular state sensor 100 and improve the wearing convenience.

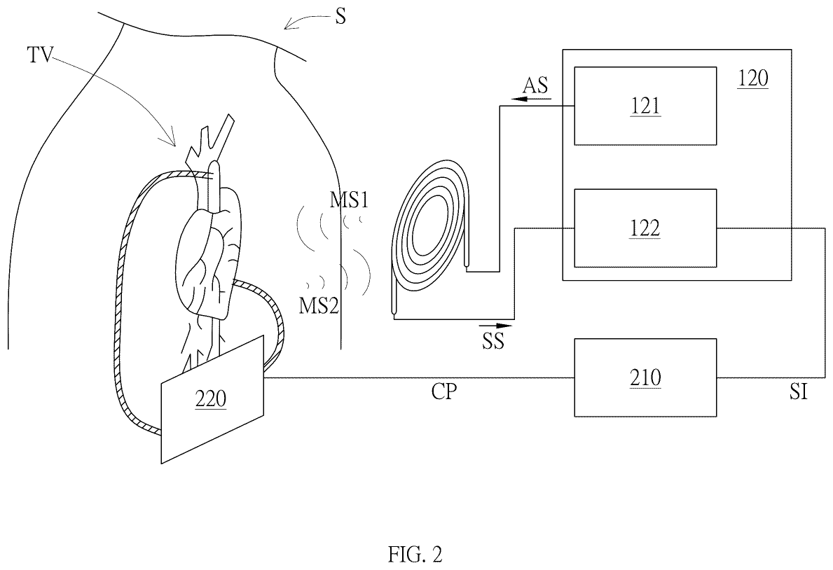

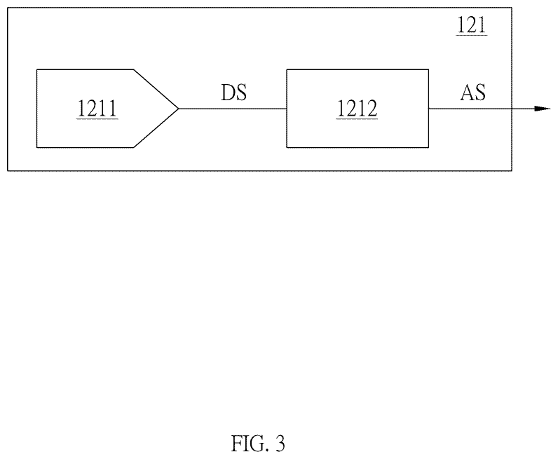

In an embodiment, as shown in FIGS. 2 and 3, the control module 120 may include the signal generating unit 121 coupled to the coil 110 and the processing unit 122. The signal generating unit 121 can be an AC/DC signal generator composed by active components (such as oscillators, and/or timers) and/or passive components (such as resistors, capacitors, and/or inductors). For example, the signal generating unit 121 may be configured to directly generate an AC signal (AS). On the other hand, the signal generating unit 121 can be configured to convert DC signal into AC signal (AS). More specifically, as shown in FIG. 3, the signal generating unit 121 includes the DC supply source 1211 and the resonant circuit 1212. The resonant circuit 1212 receives the DC signal (DS) provided by the DC supply source 1211 to generate the AC signal (AS). Because the resonant circuit 1212 only requires a series/parallel combination of passive components (such as the resistor, the capacitor, the inductor), the desired effect of generating the AC signal (AS) will be achieved by a simple circuit with low energy consumption. In the embodiment, the resonant frequency range of the resonant circuit 1212 is preferably between 1-10 MHz to correspond to the depth of the heart and achieve lower eddy current damping.

After the signal generating unit 121 provides the AC signal (AS) to the coil 110, the coil 110 generates the first electromagnetic signal (MS1) due to the electromagnetic effect. The coil 110 outputs the first electromagnetic signal (MS1) to the part to be tested (TV) (e.g. the ventricular position of the subject(S)) to induce an eddy current at the part to be tested (TV). More specifically, after the first electromagnetic signal (MS1) is applied to the part to be tested (TV), the blood at the part to be tested (TV) (blood in the heart/ventricle) can be treated as a planar conductor. Therefore, the eddy current will be induced at the planar conductor correspondingly due to the first electromagnetic signal (MS1). The amplitude, direction, frequency, and other parameters of the eddy current may be varied depending on the state of the heart/ventricle. For example, the amount of blood inside the ventricle will be varied during the ventricle systole and diastole. Different blood volumes will induce different eddy currents. The eddy current will generate the second electromagnetic signal (MS2) that is opposite to the magnetic field direction of the first electromagnetic signal (MS1). The second electromagnetic signal (MS2) will be received by the coil 110. In other words, the second electromagnetic signal (MS2) (either alone or after interacting with the first electromagnetic signal (MS1) and/or other signals) will cause the coil 110 to generate the sensing signal (SS) by the magnetoelectric effect.

The processing unit 122 of the control module 120 is configured to, for example, sample or convert, analog to digital, the sensing signal (SS), and perform calculations or measurements through components with computing capability. The processing unit 122 can perform signal analysis on the sensing signal (SS) to obtain the ventricular state information (SI) corresponding to the sensing signal (SS). For example, the processing unit 122 can determine whether the ventricle of subject(S) is in a systolic or diastolic state by analyzing the time and amplitude changes of the sensing signal (SS).

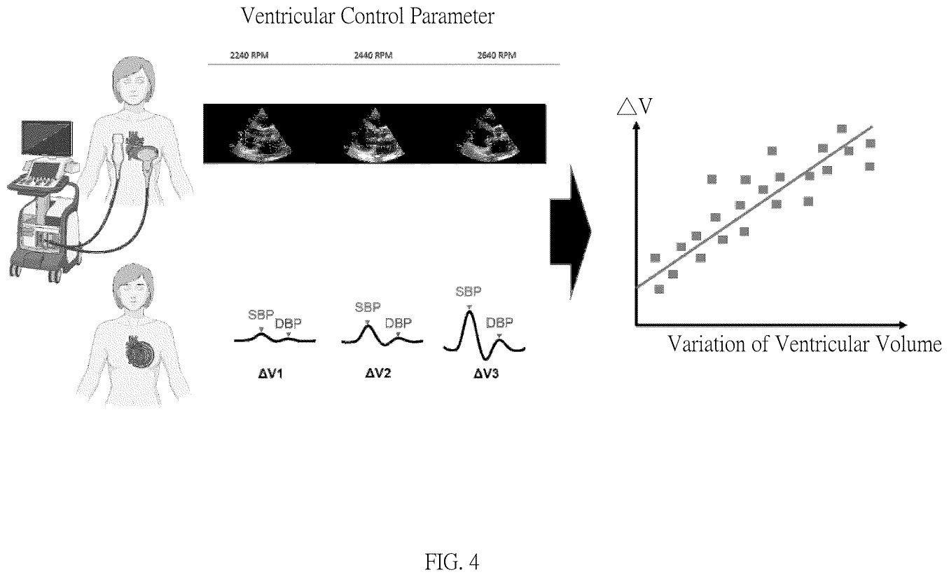

In an embodiment, as shown in FIG. 4, the correspondence relationship of the ventricular systolic/diastolic state of the subject(S) between the ventricular state sensor 100 and a conventional ultrasound system can be derived by comparing the ventricular image of the ultrasound system with the sensing signal SS measured by the ventricular state sensor 100. According to the correspondence relationship of the ventricular systolic/diastolic state of the subject(S), a database or a regression curve can be established. The correspondence relationship can be found by big data, data analysis, or AI means. In this way, when the processing unit 122 receives the sensing signal SS, the processing unit 122 can estimate the ventricular contraction/relaxation status and other ventricular state information (SI). On the other hand, it is also possible to establish a regulatory relationship between the ventricular control parameter (CP) of the ventricular assist device 200 and the contraction/relaxation of the ventricle. For example, the ventricular control parameters (CP) of the ventricular assist device 200 can be the pumping speed of the blood flow assist pump 220 or other control parameters. More specifically, when the pumping speed of the blood flow assist pump 220 is high (e.g. 2640 RPM), the volume/cross-sectional area of the diastole ventricular will be greater than the volume/cross-sectional area set at low speed (e.g. 2240 RPM). Therefore, when the ventricular control parameter (CP) is set as high speed to assist the ventricle, the signal of eddy current difference (ΔV3) between ventricular contraction and relaxation will be greater than the signal difference (ΔV1) at low speed. Therefore, the ventricular state sensor 100 can be used to monitor the ventricle which has the ventricular assist device 200 installed. The ventricular state information (SI) can be fed back to the controller 210 of the ventricular assist device 200. The controller 210 of the ventricular assist device 200 can adjust or correct the setting of the blood flow assist pump 220 based on the measurement results of the ventricular state sensor 100.

In summary, the ventricular assist system 10 can be set up to detect the ventricular contraction/relaxation through the ventricular state sensor 100 by using the eddy current sensing mechanism. The ventricular contraction/relaxation information will be fed back to the ventricular assist device 200, so that the ventricular assist device 200 can adjust the ventricular control parameter (CP) based on the subject's current state. The size and the cost of the ventricular state sensor 100 can be reduced by the electronic processes. The mechanism of the eddy current induction measurement can achieve non-invasive or preferably non-contact measurements. The ventricular assist system 10 with smaller size and lower cost also have the potential for popularization, long-term wearing, real-time measurement, and/or home use.

In an embodiment, the ventricular state sensor 100 may further include a matching component. The matching component is arranged between the coil 110 and the subject(S). More specifically, the matching component can be selected from materials with a magnetic impedance between the magnetic impedance of the subject(S) and the magnetic impedance of the coil 110. In this way, the matching component can reduce the energy loss during the energy transfer between the first electromagnetic signal (MS1) and the second electromagnetic signal MS2. Accordingly, the goal of measuring the required signal or improving the signal-to-noise ratio with less energy can be achieved. Besides, issues such as excessive energy causing injury to the subject(S) or insufficient device endurance can be avoided. On the other hand, the matching component can also serve as a contact buffer between the coil 110 and the subject(S). For example, the matching component will improve the comfort of the subject(S) or the stability during measurement. It is noted that the purpose of setting the matching component is not limited to the above examples.

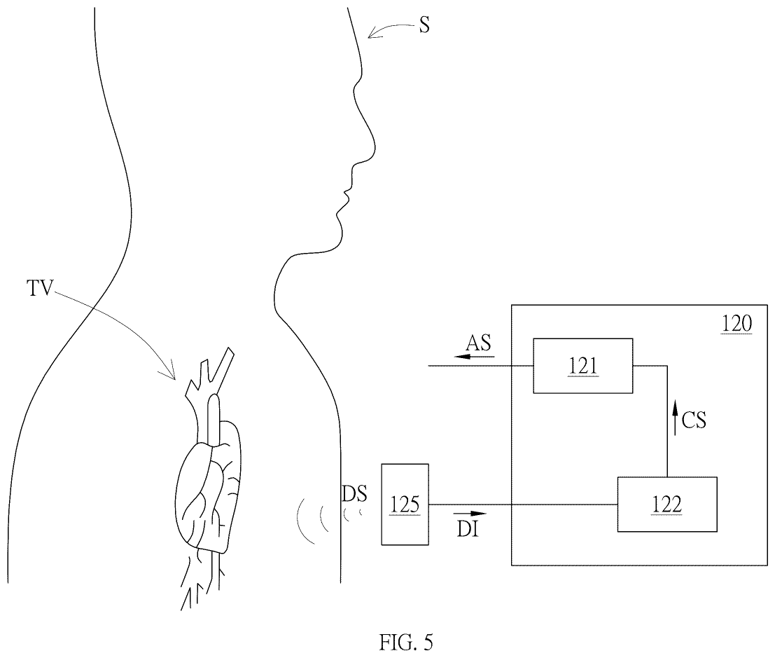

In one embodiment, referring to FIG. 5, the ventricular state sensor 100 further includes a depth detection component 125. The depth detection component 125 is configured to transmit the detection signal (DS) to the part to be tested (TV), and provide the depth information (DI) corresponding to the subject's heart to the control module 120. The control module 120 adjusts the frequency or intensity of the first electromagnetic signal (MS1) based on the depth information (DI). For example, the processing unit 122 of the control module 120 may receive the depth information (DI) and generate a control signal (CS). The control signal (CS) is configured to control the signal generating unit 121 to adjust, based on the depth information (DI), the frequency or amplitude of the AC signal (AS) provided to the coil 110. The coil 110 receiving the AC signal (AS) will generate the first electromagnetic signal (MS1) with different frequencies and/or strengths. Alternatively, the first electromagnetic signal (MS1) can be adjusted by adjusting the electrical characteristics of the coil 110 (such as impedance value, inductance value, capacitance value) by the control signal (CS). However, the means for adjusting the first electromagnetic signal (MS1) is not limited to the examples mentioned above. On the other hand, the depth detection component 125 can be a component with a detection means which can pass through the intermedia (such as penetrating skin, fabric, or other media) such as an optics component (detecting signal as light signals) or an acoustics component (detecting signal as sound waves). The depth detection component 125 may measure the depth of the heart of the subject through ranging mechanisms such as time-of-flight (TOF). The components and mechanisms for measuring depth information (DI) are not limited to the examples mentioned above.

For the subjects(S) with different weights or conditions, the depth detection component 125 will provide the depth information (DI), hence, the control module 120 will accurately know the distance between the heart and the coil 110. Accordingly, the optimal signal frequency for measurement can be selected to improve the energy transfer efficiency of the first electromagnetic signal (MS1) and/or the second electromagnetic signal MS2. The depth detection component 125 may reduce the power loss of the ventricular state sensor. Besides, an efficiency transferring method will significantly reduce the risk of subject(S) being exposed to the electromagnetic waves.

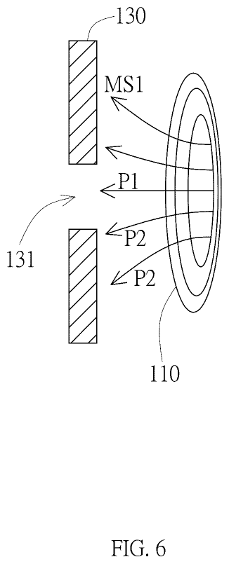

In an embodiment, referring to FIG. 6, the ventricular state sensor further includes the isolation unit 130. The isolation unit 130 is arranged between the coil 110 and the subject(S). The isolation unit 130 has the gap 131. The first part (P1) of the first electromagnetic signal (MS1) toward to the part to be tested (TV) of the subject(S) is allowed to pass through the gap 131. In other words, the first part (P1) of the first electromagnetic signal (MS1) is not blocked by the isolation unit 130. The isolation unit 130 only blocks the second part (P2) of the first electromagnetic signal (MS1). More specifically, the second part (P2) which is not directed to the part to be tested (TV) of the subject(S) is blocked by the isolation unit 130 and will not induce the eddy current at the part to be tested (TV) of the subject(S).

The material of the isolation unit 130 can be an electrical conductor, magnetic conductor, or other materials that can block electromagnetic waves. The first electromagnetic signal (MS1) is emitted from the coil 110 toward the part to be tested (TV). The first electromagnetic signal (MS1) still has the second part that diverges and does not aim to the part to be tested (TV) due to the divergence of magnetic field. Therefore, the second part (P2) of the first electromagnetic signal (MS1) does not fully (or cannot) act on the part to be tested (TV), and may generate noise. The generated noise may interfere with the first part (P1) of the first electromagnetic signal (MS1) that does not diverge and is directed towards the part to be tested (TV) and/or the second electromagnetic signal (MS2) generated in response to the eddy current (I). In this embodiment, through the gap 131 of the isolation unit 130, the first part (P1) of the first electromagnetic signal (MS1) will pass through the isolation unit 130 without being shielded by the isolation unit 130, while the second part (P2) of the first electromagnetic signal (MS1) will be shielded by the isolation unit 130. By setting the isolation unit 130, the goal of improving signal-to-noise ratio can be achieved. In addition, the shape of the gap 131 may be selected from circular, square, or other shapes based on the shape of the coil 110. It should be noted that the shape and/or arranging location of the gap can be adjusted according to needs.

By using the isolation unit 130, the measurement of the ventricular state sensor can be more directional and anti-interference. This can improve the measurement quality of the ventricular state sensor (such as improving the signal-to-noise ratio). With more directional measurement, the total energy to induce the eddy current at the part to be tested (TV) will be reduced. Therefore, the risk of subject(S) being exposed to the electromagnetic waves will be reduced. Besides, the impact of scattered electromagnetic waves on the operation of other devices will be avoided.

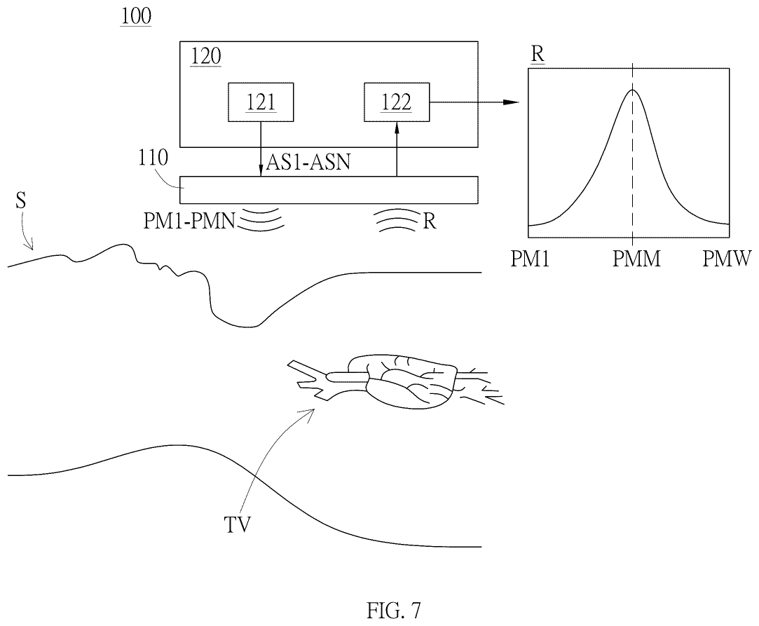

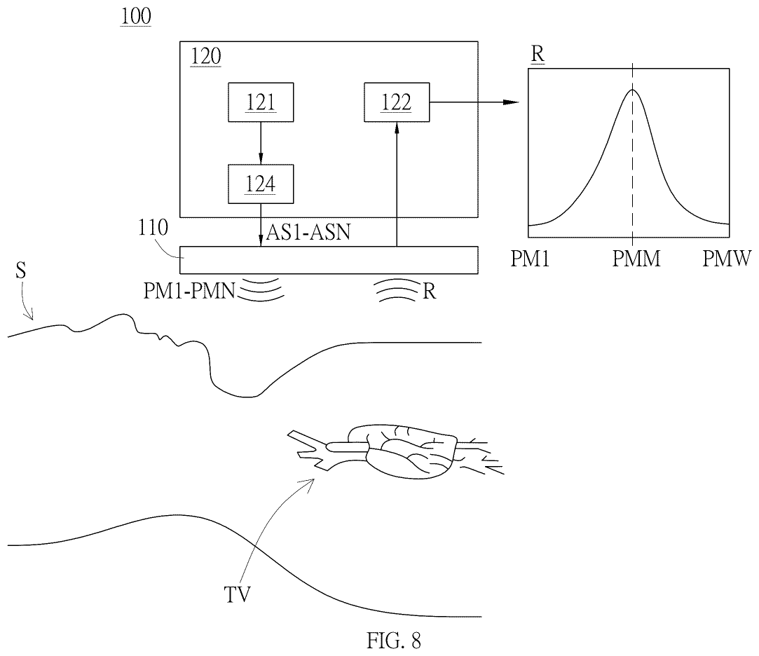

In an embodiment, as shown in FIGS. 7 and 8, the coil 110 of the ventricular state sensor outputs at least one leading electromagnetic signal (e.g. PM1-PMN) before outputting the first electromagnetic signal (MS1). Each leading electromagnetic signal (e.g. PM1-PMN) corresponds to one signal parameter of a parameter group, and the signal parameter of the first electromagnetic signal (MS1) will correspond to the one of the leading electromagnetic signals (PM1-PMN) with optimal response.

More specifically, the coil 110 generates the first electromagnetic signal (MS1) based on the AC signal (AS) provided by the signal generating unit 121. However, for different subjects or subjects of different wearing forms, signal parameters (such as frequency, amplitude, intensity) of the first electromagnetic signal (MS1) should be adjusted to obtain the best/better measurement results. Therefore, before outputting the first electromagnetic signal (MS1) from the coil 110, a scan is performed by transmitting the at least one leading electromagnetic signal (e.g. PM1-PMN) to indicate the optimal or relatively optimal first electromagnetic signal (MS1) for measuring different subjects.

In the embodiment, as shown in FIG. 7, one or more leading AC signals (e.g. AS1-ASN) can be output from the signal generating unit 121 to the coil 110 to generate one or more leading electromagnetic signals (e.g. PM1-PMN). Each of the leading electromagnetic signals (PM1-PMN) corresponds to one of the leading AC signals (AS1-ASN). More specifically, the signal generating unit 121 outputs the leading AC signals (AS1-ASN) with different frequencies in sequence (e.g., in increasing or in decreasing) within a desired frequency range (e.g., from kilohertz to megahertz). By doing so, the leading electromagnetic signals (PM1-PMN) can be generated. Each frequency in the leading electromagnetic signals (PM1-PMN) will correspond to one of the frequencies in the leading AC signals (AS1-ASN). By scanning with the leading electromagnetic signals (PM1-PMN) of different frequencies, the part to be tested (TV) will be induced to generate eddy currents with different responses. For example, different intensities of response electromagnetic signals may be generated. The control module 120 can determine the frequency of the AC signal (AS) output by the signal generating unit 121 based on the transmission frequency corresponding to the maximum or optimal response (PMM) received by the coil 110. Accordingly, the coil 110 will generate the optimal or relatively optimal first electromagnetic signal (MS1) for measurement.

In the embodiment, another mechanism for regulating the signal parameters of the leading electromagnetic signals (PM1-PMN) is shown in FIG. 8. The control module 120 includes an adjustable passive component 124 coupled to the coil 110. The control module 120 adjusts the capacitance value, inductance value, and/or impedance value of the adjustable passive component 124 to adjust the signal parameters of the at least one leading electromagnetic signals (e.g. PM1-PMN). More specifically, the adjustable passive component 124 coupled to the coil 110 can be, for example, an adjustable capacitor. Adjusting the capacitance value of the adjustable capacitor can indirectly adjust the capacitance and/or impedance value of the coil 110. Therefore, when the coil 110 generates the leading electromagnetic signals (PM1-PMN), the signal parameters of each of the leading electromagnetic signals (PM1-PMN) can be different by changing the capacitance value of the adjustable passive component 124 with the same AC signal (AS) provided by the signal generating unit 121. By using the leading electromagnetic signals (PM1-PMN), different responses of eddy currents are generated at the part to be tested (TV). The leading electromagnetic signals (PM1-PMN) will indicate the optimal/relatively optimal capacitance value combination of the coil 110 and the adjustable passive component 124. It should be noted that the adjustable passive component 124 is not limited to adjusting capacitance values. The adjustable passive component 124 can be any components which is able to adjust at least one of the values of resistance, impedance, capacitance, and/or inductance.

By using the leading electromagnetic signals (PM1-PMN), the ventricular state sensor 100 may perform a pre-scan process for calibration of the first electromagnetic signal (MS1). Thereby generating the most suitable measurement parameters for different subjects or different tested positions will be achieved. The pre-scan will avoid measurement errors caused by individual differences of subjects or different the tested positions. The pre-scan indicates better measurement parameters and improves the measurement efficiency, which will reduce the power loss of the ventricular state sensor and reduce the risk of exposing to electromagnetic waves.

The previous description of the present invention is provided to enable a person of ordinary skill in the art to make or implement the present invention. Various modifications to the present invention will be apparent to a person skilled in the art, and the general principles defined herein can be applied to other variations without departing from the spirit or scope of the present invention. Therefore, the present invention is not intended to be limited to the examples described herein, but is to be in accord with the widest scope consistent with the principles and novel features of the invention herein.

What is claimed is:

1. A ventricular assist system, comprising:
a ventricular state sensor including:
a coil; and
a control module coupled to the coil, wherein the control module is configured to drive the coil to perform an eddy current induction measurement on a ventricle of a subject, and derive at least one ventricular state information of the subject through the eddy current induction measurement; and
a ventricular assist device coupled to the ventricular state sensor, wherein the ventricular assist device receives the at least one ventricular state information to adjust at least one ventricular control parameter of the ventricular assist device.

2. The ventricular assist system of claim 1, wherein the eddy current induction measurement includes:
transmitting a first electromagnetic signal by the coil to the ventricle of the subject; and
receiving a second electromagnetic signal generated by inducing the first electromagnetic signal from the ventricle of the subject.

3. The ventricular assist system of claim 2, wherein the control module calculates the at least one ventricular state information according to the first electromagnetic signal and the second electromagnetic signal.

4. The ventricular assist system of claim 1, wherein the control module includes:
a signal generating unit coupled to the coil and configured to generate an AC signal and provide the AC signal to the coil to generate a first electromagnetic signal; and
a processing unit coupled to the coil, wherein the processing unit receives a sensing signal from the coil and calculates the at least one ventricular state information according to the sensing signal.

5. The ventricular assist system of claim 1, wherein the at least one ventricular control parameter includes a pumping speed of a blood flow assist pump.

6. The ventricular assist system of claim 1, wherein before the coil performs the eddy current induction measurement, the coil further outputs at least one leading electromagnetic signal; each of the at least one leading electromagnetic signal corresponds to a signal parameter; a signal parameter of a first electromagnetic signal for the eddy current induction measurement corresponds to one of the at least one leading electromagnetic signal with an optimal response.

7. The ventricular assist system of claim 6, wherein the control module includes an adjustable passive component coupled to the coil; the control module adjusts the adjustable passive component to adjust the signal parameter of each of the at least one leading electromagnetic signal.

8. The ventricular assist system of claim 1, wherein the ventricular state sensor further includes:
an isolation unit arranged between the coil and the subject, wherein the isolation unit has a gap for allowing a first portion of a first electromagnetic signal directed toward the ventricle of the subject to pass therethrough, and the isolation unit is configured to block a second portion of the first electromagnetic signal not passing the gap.

9. The ventricular assist system of claim 1, wherein the ventricular state sensor further includes:
a depth detection component configured to output a detection signal to the ventricle of the subject and provide a depth information corresponding to the ventricle of the subject to the control module.

10. The ventricular assist system of claim 1, wherein the ventricular state sensor further includes:
a matching component arranged between the coil and the subject, wherein a magnetic impedance of the matching component is selected between a magnetic impedance of the subject and a magnetic impedance of the coil.
